(19) United States
(12) Patent Application Publication
(10) Pub. No.: US 2005/0143363 A1
(43) Pub. Date: Jun. 30, 2005

(54) Method for subretinal administration of therapeutics including steroids; method for localizing pharmacodynamic action at the choroid of the retina; and related methods for treatment and/or prevention of retinal diseases

(75) Inventors: Eugene De Juan, La Canada Flintridge, CA (US); Signe Varner, Los Angeles, CA (US)

Correspondence Address: KAGAN BINDER, PLLC, SUITE 200, MAPLE ISLAND BUILDING, 221 MAIN STREET NORTH, STILLWATER, MN, 55082, US

(73) Assignee: InnoRx, Inc.

(21) Appl. No.: 10/507,461

(22) PCT Filed: Sep. 29, 2003
(86) PCT No.: PCT/US03/30716

Related U.S. Application Data
(60) Provisional application No. 60/414,782, filed on Sep. 29, 2002.
(60) Provisional application No. 60/467,291, filed on May 2, 2003.

(51) Int. Cl. A61K 31/573

(52) U.S. Cl. 514/179

(57) ABSTRACT

Featured is a methodology for administering a therapeutic medium to the posterior segment of an eye including instilling or disposing the therapeutic medium sub-retinally. In particular embodiments, the therapeutic medium is disposed in a sub retinal space. Such instillation being accomplished by one of injection or implantation of the therapeutic medium sub-retinally or the sub-retinal space. In other aspects, the methodology further includes forming a limited retinal detachment so as to define the sub-retinal space as well as methods for treating an eye by sub-retinally administering a therapeutic medium.

This application claims the benefit of U.S. Provisional Application Ser. No. 60/414,782 filed Sep. 29, 2002 and U.S. Provisional Application Ser. No. 60/467,291 filed May 2, 2003, the teachings of each being incorporated herein by reference.

### FIELD OF INVENTION

The present invention relates to methods and techniques for treating eyes, such as eyes of mammals having eye disorders or diseases, more particularly to methods and techniques for administering a therapeutic medium or agent such as steroids sub-retinally, more specifically, to methods and techniques for administering such therapeutics or agents to the tissues of the eye so that the pharamacodynamic action of the such therapeutics/agents is localized at the choroid and the retina. Also featured are methods related thereto for treating eyes using such therapeutics or agents and prophylactic administration of such therapeutics to eyes.

### BACKGROUND OF THE INVENTION

There are a number of vision-threatening disorders or diseases of the eye of a mammal including, but not limited to diseases of the retina, retinal pigment epithelium (RPE) and choroid. Such vision threatening diseases include, for example, ocular neovascularization, ocular inflammation and retinal degenerations. Specific examples of these disease states include diabetic retinopathy, chronic glaucoma, retinal detachment, sickle cell retinopathy, age-related macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases, retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury.

For example, age-related macular degeneration (AMD) is the leading cause of irreversible severe central vision loss in Caucasians fifty years old and older in the United States. According to the 1990 U.S. census, approximately 750,000 people over 65 years of age were estimated as severe visual impairment in one or both eyes from AMD. Also, the number of cases of AMD has been predicted to increase from 2.7 million in 1970 to 7.5 million by the year 2030.

Roughly 80 percent of the AMD cases involve non-neovascular conditions, for which there are no effective treatments. For the remaining cases involving neovascularization, currently available treatments are sub-optimal. Perhaps the best known therapy is photodynamic therapy (PDT), however, while this therapy has received significant intention in both the ophthalmic and financial investment communities, it is useful in only about 20 percent of neovascular AMD cases. In addition, this particular therapy is not a simple or inexpensive treatment. The procedure generally needs to be repeated every three months for at least two years, with approximate total cost of $12,250.

A number of angiostatic agents are currently under investigation for the treatment of AMD. Thalidomide, for example, is known to be a powerful angiostatic agent. It systemic side effects, however, include peripheral neuropathy, central nervous system depression, and embryotoxicity. In addition, these systemic side effects have limited the dosage administered to patients for the treatment of sub-retinal neovascularization. Systemic inhibition of angiogenesis in older patients can also interfere with the development of collateral circulation, which has a role in the prevention of central nervous system as well as cardiac ischemic events.

A number of techniques or methodologies have been developed to deliver drugs to the various tissues or structure that make up the mammalian eye as described hereinafter to treat a wide range of disorders or diseases of the eye. However, delivery of drugs, proteins and the like to the eye(s) of mammals so as to achieve the desired therapeutic or medical effect, especially to the retina and/or the choroids, has proven to be challenging, most of which is owed to the geometry, delicacy and/or behavior of the eye and its components. A brief description of various conventional methods or techniques for delivering drugs to the tissues of the eye and the shortcomings thereof are hereinafter described.

Oral ingestion of a drug or injection of a drug at a site other than the eye can provide a drug systemically, however, such a systemic administration does not provide effective levels of the drug specifically to the eye. In many ophthalmic disorders involving the retina, posterior tract, and optic nerve, adequate levels of the drug cannot be achieved or maintained by oral or parenteral routes of administration. Thus, further and repeated administration of the drug would be necessary to achieve the desired or adequate levels of concentration of the drug. Such further and repeated administrations of such drugs, however, may produce undesired systemic toxicity.

Ophthalmic conditions have also been treated using drugs applied directly to the eye in either liquid or ointment form. This route of administration (i.e., topical administration), however, is only effective in treating problems involving the superficial surface of the eye and diseases that involve the cornea and anterior segment of the eye, such as for example, conjunctivitis. Topical administration of drugs is ineffective in achieving adequate concentrations of a drug(s) in the sclera, vitreous, or posterior segment of the eye. In addition, topical eye drops may drain from the eye through the nasolacrimal duct and into the systemic circulation, further diluting the medication and risking unwanted systemic side effects. Furthermore, delivery of drugs in the form of topical eye drops is also of little utility because the drug cannot cross the cornea and be made available to the vitreous, retina, or other subretinal structures such as the retinal pigment epithelium (“RPE”) or choroidal vasculature and/or is highly unstable and therefore not easily formulated for topical delivery. Moreover, data also indicates that it is not unusual for up to 85% of topically applied agents to be removed by the eye's blink mechanism/reflex.

Direct delivery of drugs to the eye by a topical insert has also been attempted, however, this method is not desirable. Such topical inserts require patient self-administration and thus education on their insertion into and removal from the eye. Consequently, this technique demands a certain degree of manual dexterity that can be problematic for geriatric patients who are particularly susceptible to certain eye disorders that appear age related (e.g., age related macular degeneration). Also, in many instances such topical inserts may cause eye irritation and such inserts are prone to inadvertent loss due to eyelid laxity. In addition, these devices provide a source of drug only to the cornea and anterior chamber, and thus do not provide any pharmacologic advantage over topical eye drops or ointments. Thus, such devices have limited, if any at all, utility for providing an effective source of drugs to the vitreous or tissues located in the posterior segment of the eye.

As a consequence most methods for treating eye disorders or diseases in the posterior segment, or the back-of-the-eye, involve intravitreal deliver of the drug. One such technique for intravitreal delivery is accomplished by intraocular injection of the drug or microspheres containing the drug directly into the vitreous or by locating a device or capsule containing the drug in the vitreous, such as that described in U.S. Pat. No. 5,770,589. Intravitreal injection of a drug is an effective means of delivering the drug to the posterior segment of the eye in high concentrations, but it is not without its shortcomings. It is well known that drugs that are initially located within the vitreous are removed from the vitreous over time via the anterior segment of the eye. If the ocular condition is anything other than acute, this technique necessarily requires follow-up injections in order to maintain an adequate therapeutic concentration within the vitreous. This, in turn, presents problems because each additional intraocular injection carries with it a realistic risk of infection, hemorrhage and/or retinal detachment.

In addition, it also is well known that many therapeutic drugs cannot easily diffuse across the retina. Thus, the dose being administered and maintained in the vitreous has to take into account the amount that can diffuse across the retinal boundary as well as how long the drug is retained in effective amounts within the vitreous. For example, it has been observed from animal studies that 72 hours after injection of triamcinolone, less than 1% of the triamcinolone present in the vitreous was associated with other tissues including the retina, pigment epithelium, and sclera. In addition to the relative effectiveness of drug delivery across the barrier, complications or side effects have been observed when using the direct injection into vitreous technique with some therapeutics.

For example, compounds classified as corticosteroids, such as triamcinolone, can effectively treat some forms of neovascularization such as corneal neovasularization. When these compounds were used to treat neovscularization of the posterior segment by direct injection, these compounds were observed to cause undesirable side effects in many patients. The adverse affects or undesirable side effects being observed included elevations in intraocular pressure and the formation of, or acceleration of, the development of cataracts. Elevations in intraocular pressure are of particular concern in patients who are already suffering from elevated intraocular pressure, such as glaucoma patients. Moreover, a risk exists that the use of corticosteroids in patients with normal intraocular pressure will cause elevations in pressure that result in damage to ocular tissue. Since therapy with corticosteroids is frequently long term, i.e., several days or more, a potential exists for significant damage to ocular tissue as a result of prolonged elevations in intraocular pressure attributable to that therapy.

Consequently, efforts in the area of intravitreal delivery also have included delivery by locating a sustained release implant, capsule or other such device or mechanism that is in communication with the vitreous and which is configured so as to provide a release over time into the vitreous of the contained drug. Examples of such controlled release devices are described in U.S. Pat. No. 6,217,895; U.S. Pat. No. 5,773,019; U.S. Pat. No. 5,378,475 and U.S. Patent Application Publication No. 2002/0061327.

A common feature of the techniques/instruments described therein, is that a surgical incision is required to be made at the outset of a procedure so that the implant, capsule or other such device can be inserted through the eye and located in the vitreous. These methods and techniques also necessarily involve the use of sutures following completion of the procedure to seal or close the incision so as to prevent loss of vitreous material. As is known to those skilled in the art, maintaining the volume of the posterior segment or vitreous is necessary to maintaining the shape and optical arrangement of the eye. Such a course of treatment also increases the duration and cost as well as the realistic risks of corneal ulceration, cataract formation, intraocular infection, and/or vitreous loss that accompany these procedures.

There is described in U.S. Pat. Nos. 5,273,530 and 5,409,457 an instrument and methodology to transplant donor cells, more specifically donor retina cells, in the sub-retinal space. It also is described therein that the instrument also can be used to inject or remove material from the vitreous. According to the described methodology, the instrument is shaped and dimensioned so it can be inserted into an eye orbit along an insertion path that extends along the periphery of the eye and so as to place the tip adjacent to the retina or sub-retinal region. The tip is then moved generally in the medial direction so the tip pierces the exterior of the eye and so the tip resides in the sub-retinal region or in the vitreous depending upon how much the tip is moved. In order to prevent over-insertion of the tip, a collar is provided about the tip so as to limit the distance the tip can be inserted into the eye.

There also is described in U.S. Patent Application Publication 2002/0055724, an instrument for sub-retinal transplantation of retinal cells, epithelium and choroid within their normal planar configuration as a graft into the sub-retinal region of an eye. The described instrument is inserted into an opening in the eye using either a transcorneal surgical approach or a trans-choroidal and scleral surgical approach. According to this technique the instrument is advanced under the retina to detach the retina so that the graft can be inserted. As noted in U.S. Pat. No. 5,273,530, the penetration of the anterior part or segment of the eye, using the transcorneal or the transscleral route creates the risks of corneal ulceration, cataract formation and other anterior penetration problems. Also using either approach, a surgical incision is created at the outset of a procedure so that the instrument can be inserted and sutures are used following completion of the procedure to seal or close the incision so as to prevent loss of vitreous material (i.e., aqueous humor).

It thus would be desirable to provide methods for treating an eye, particularly treating retinal and/or choridal disorders or diseases, by locating a depot of a therapeutic medium, compound or agent such as a corticosteriod, in the sub-retinal space of the eye. It would be particularly desirable to provide such a method that would localize the action of the therapeutic medium, compound or agent (e.g., anti-inflammatory steroid, corticosteriod) at the retina and the choroidea while minimizing such action in other tissues of the eye.

### SUMMARY OF THE INVENTION

The present invention features methods for administering or delivering a therapeutic medium to a posterior segment of a mammalian eye, more particularly a human eye, where such a therapeutic medium includes, but is not limited to drugs, medicaments, antibiotics, antibacterials, antiproliferatives, neuroprotectives, anti-inflammatories (steroidal and non-sterodial), growth factors, neurotropic factors, antiangiogenics, thromobolytics or genes. The present invention also features methods for the treatment and prevention of disorders and or diseases of the eye, in particular retinal/choroidal disorders or diseases, through sub-retinal administration or sub-retinal prophylatic administration of such a therapeutic medium. More particularly, such methods according to the present invention include instilling or disposing a therapeutic amount of a therapeutic medium sub-retinally or into the sub-retinal space, more specifically so as to localize the action of the therapeutic medium at the choroid and the retina of the eye. In a more particular embodiment, said instilling or disposing includes injecting or implanting such a therapeutic medium sub-retinally or in the sub-retinal space.

Such methods bypass the mechanisms that limit effective delivery of therapeutic media to the retina/choriod when they are injected directly into the vitreous, thereby permitting more sustained therapy for the target tissue. Moreover, locating such a therapeutic medium sub-retinally or in the sub-retinal space also reduces the side effects typically associated with the injection of drugs into the vitreous.

Exemplary therapeutic mediums include, but are not limited to, thrombin inhibitors; antithrombogenic agents; thrombolytic agents; fibrinolytic agents; vasospasm inhibitors; calcium channel blockers; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil); immunological drugs (such as vaccines and immune stimulants); hormonal agents (such as estrogens, estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotropin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Antiproliferatives include any of a number of compounds, agents, therapeutic mediums or drugs known to those skilled in the art that inhibit the proliferation of cells. Such compounds, agents, therapeutic mediums or drugs include, but are not limited to, 5-fluorouracil, taxol, rapamycin, mitomycin C and cisplatin.

Neuroprotectives include any of a number of compounds, agents, therapeutic mediums or drugs known to those skilled in the art that guard or protect against neurotoxicity; the quality of exerting a destructive or poisonous effect upon nerve tissue. Such compounds, agents, therapeutic mediums or drugs include, but are not limited to, lubezole.

Anti-inflammatories include any of a number of compounds, agents, therapeutic mediums or drugs known to those skilled in the art, either steroidal or non-steroidal, and generally characterized has having the property of counteracting or suppressing the inflammatory process. Non-steroidal inflammatory drugs or compounds comprise a class of drugs which shares the property of being analgesic, antipyretic and anti-inflammatory by way of interfering with the synthesis of prostaglandins. Such non-steroidal anti-inflammatories include, but are not limited to, indomethacin, ibuprofen, naxopren, piroxicam and nabumetone.

Such anti-inflammatory steroids contemplated for use in the methodology of the present invention, include those illustrated in FIGS. 6A-C and also that described in U.S. Pat. No. 5,770,589, the teachings of which are incorporated herein by reference. In an exemplary embodiment, an anti-inflammatory steroid contemplated for use in the methodology of the present invention is triamcinolone acetonide (generic name). Corticosteroids contemplated for use in the methodology of the present invention include, for example, triamcinolone, dexamethasone, fluocinolone, cortisone, prednisolone, flumetholone, and derivatives thereof (See also U.S. Pat. No. 5,770,589, the teachings of which are incorporated herein by reference).

As is known to those skilled in the art, growth factors is a collective term originally used to refer to substances that promote cell growth and is now loosely used to describe molecules that function as growth stimulators (mitogens) but also as growth inhibitors (sometimes referred to as negative growth factors), factors that stimulate cell migration, or as chemotactic agents or inhibit cell migration or invasion of tumor cells, factors that modulate differentiated functions of cells, factors involved in apoptosis, factors involved in angiogenesis, or factors that promote survival of cells without influencing growth and differentiation. In the present invention, such growth factors include, but are not limited to, pigment epithelium derived factor and basic fibroblast growth factor.

As is known to those skilled in the art, neurotropic factors is a general term used to describe growth factors and cytokines that can enhance neuronal survival and axonal growth and that regulate synaptic development and plasticity in the nervous system. In the present invention, such growth factors include, but are not limited to, ciliary neurotrophic factors and brain-derived neurotrophic factors.

Antiangiogenics include any of a number of compounds, agents, therapeutic mediums or drugs known to those skilled in the art that inhibit the growth and production of blood vessels, including capillaries. Such compounds, agents, therapeutic mediums or drugs include, but are not limited to, anecortave acetate and anti VEGF antibody.

Thrombolytics, as is known to those skilled in the art include any of a number of compounds, agents, therapeutic mediums or drugs that dissolve blot clots, or dissolve or split up a thrombus. Such thrombolytics include, but are not limited to, streptokinase, tissue plasminogen activator or TPA and urokinase.

The therapeutic medium being instilled or disposed sub-retinally or in the sub-retinal space is in any of a number of formulations including fluid solutions, solids and/or sustained release formulations or devices. In an even more particular embodiment, such instilling or disposing includes forming a local or limited retinal detachment so as to define a sub-retinal space and injecting and/or implanting the therapeutic medium, in what ever form, into the sub-retinal space defined by the local/limited retinal detachment.

In further embodiments, sustained releases devices of the present invention include, but are not limited to those having the following characteristics; flexible rods, thin films, foldable discs, biodegradable polymers with the therapeutic medium (e.g., drug) embedded within, drug eluting polymer coatings over a rigid scaffold, compressed drug “pellets” or a therapeutic medium encapsulated in a semi-permeable membrane. Also, some characteristic formulations for delivery of the therapeutic medium into the subretinal space include, but are not limited to, injectable hydrogels, cyclodextrin “solubilized” and micronized solutions.

A variety of biocompatible capsules are suitable for delivery of the therapeutic medium. Exemplary biocompatible polymer capsules contemplated for use in the methodology of the present invention comprise (a) a core which contains the therapeutic medium, either suspended in a liquid medium or immobilized within a biocompatible matrix, and (b) a surrounding jacket comprising a membrane that is biocompatible and permits diffusion of the drugs, therapeutics, medicaments such as proteins, cells or small molecule pharmaceuticals, or the like to the tissues proximal the sub-retinal space. As indicated above, the core may comprise a biocompatible matrix of a hydrogel or other biocompatible matrix material that stabilizes the position of the therapeutic medium. The jacket for the capsule may be manufactured from various polymers and polymer blends including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers, and mixtures thereof.

Most, if not all, ophthalmic diseases and disorders are associated with one or more of three types of indications: (1) angiogenesis, (2) inflammation, and (3) degeneration. Based on the indications of a particular disorder, one of ordinary skill in the art can administer any suitable therapeutic medium molecule from the three groups at a therapeutic dosage. The following describes some ophthalmic diseases and disorders and a form of treatment therefore. It should be recognized however, that the following is by way of illustration and is not intended to limit the methodologies of the present invention to a particular technique or therapeutic medium for treatment of an eye disease or disorder.

Diabetic retinopathy, for example, is characterized by angiogenesis. This invention contemplates treating diabetic retinopathy by delivering one or more anti-angiogenic factors into the sub-retinal space. It also is desirable to co-deliver one or more neurotrophic factors also to the sub-retinal space.

Uveitis involves inflammation. The present invention contemplates treating uveitis by instilling or disposing one or more anti-inflammatory factors in the sub-retinal space.

Retinitis pigmentosa, by comparison, is characterized by retinal degeneration. The present invention contemplates treating retinitis pigmentosa by instilling or disposing one or more neurotrophic factors in the sub-retinal space.

Age-related macular degeneration involves both angiogenesis and retinal degeneration and includes, but is not limited to, dry age-related macular degeneration, exudative age-related macular degeneration, and myopic degeneration. The present invention contemplates treating this disorder by instilling or disposing in the sub-retinal space one or more neurotrophic factors and/or one or more anti-angiogenic. More particularly, the methodology contemplates instilling or disposing a corticosteriod in the sub-retinal space.

Glaucoma is characterized by increased ocular pressure and loss of retinal ganglion cells. Treatments for glaucoma contemplated in the present invention include delivery of one or more neuroprotective agents that protect cells from excitotoxic damage. Such agents include N-methyl-D-aspartate (NMDA) antagonists, cytokines, and neurotrophic factors.

Other aspects, embodiments and advantages of the present invention will become readily apparent to those skilled in the art are discussed below. As will be realized, the present invention is capable of other and different embodiments without departing from the present invention. Thus the following description as well as any drawings appended hereto shall be regarded as being illustrative in nature and not restrictive.

### DEFINITIONS

The instant invention is most clearly understood with reference to the following definitions:

Vitreous shall be understood to mean the vitreous or vitreal cavity of a mammalian eye.

Aqueous of the eye shall be understood to mean the aqueous humor of the eye.

Sustained release device shall be understood to mean any of a number of devices that are configured and arranged to release a drug(s) over an extended period of time in a controlled fashion.

The term “hydrogel” shall be understood to mean a three dimensional network of cross-linked hydrophilic polymers. The network is in the form of a gel, substantially composed of water, preferably gels being greater than 90% water.

As used herein, “therapeutically effective amount” refers to that amount of a therapeutic medium alone, or together with other substances, that produces the desired effect (such as treatment of a medical condition such as a disease or the like, or alleviation of pain) in a patient. During treatment, such amounts will depend upon such factors as the particular condition being treated, the severity of the condition, the individual patient parameters including age, physical condition, size and weight, the duration of the treatment, the nature of the particular bioactive agent thereof employed and the concurrent therapy (if any), and like factors within the knowledge and expertise of the health practitioner. A physician or veterinarian of ordinary skill can readily determine and prescribe the effective amount of the therapeutic medium required to treat and/or prevent the progress of the condition.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a flow diagram of methodology for administering or delivering a therapeutic according to an embodiment of the present invention;

FIG. 2 is a flow diagram of methodology for administering or delivering a therapeutic according to another embodiment of the present invention;

FIGS. 3A,B are illustrative views of the sub-retinal drug devices described in U.S. Ser. No. 09/888,079;

FIGS. 4A,B are illustrative views illustrating the localization of the operable end of a sub-retinal drug delivery device of FIG. 3A;

FIG. 5 is an axonometric view of an exemplary operable end of a sub-retinal delivery device having a delivery cannula for delivering the therapeutic medium to the sub-retinal space;

FIGS. 6A-C are formulas illustrative of steroidal anti-inflammatories contemplated for use with the methodologies of the present invention.

FIG. 7 is a flow diagram of methodology for administering or delivering a therapeutic according to yet another embodiment of the present invention; and

FIG. 8 is a flow diagram of methodology for administering or delivering a therapeutic according to yet another embodiment of the present invention.

### DESCRIPTION OF THE PREFERRED EMBODIMENT

The present invention provides a methodology for sub-retinal administration or delivery of a therapeutic medium to a posterior segment of a mammalian eye, more particularly a human eye as well as a methodology for treating and/or preventing disorders and/or diseases of the eye, in particular retinal/choroidal disorders or diseases, through such sub-retinal administration of such therapeutic mediums. Such methodologies provide a mechanism for treating a wide array of diseases and/or disorders of an eye of a mammal, more specifically a human eye, and more particularly diseases or disorders involving the posterior segment of the eye such as retinal/choroidal disorders or diseases. Such a treatment/prevention methodology also is useable to treat/prevent a number of vision-threatening disorders or diseases of the eye of a mammal including, but not limited to diseases of the retina, retinal pigment epithelium (RPE) and choroid. Such vision threatening diseases include, for example, ocular neovascularization, ocular inflammation and retinal degenerations. Specific examples of these disease states include diabetic retinopathy, chronic glaucoma, retinal detachment, sickle cell retinopathy, age-related macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury. The methodology of the present invention also can be used to treat ocular symptoms resulting from diseases or conditions that have both ocular and non-ocular symptoms.

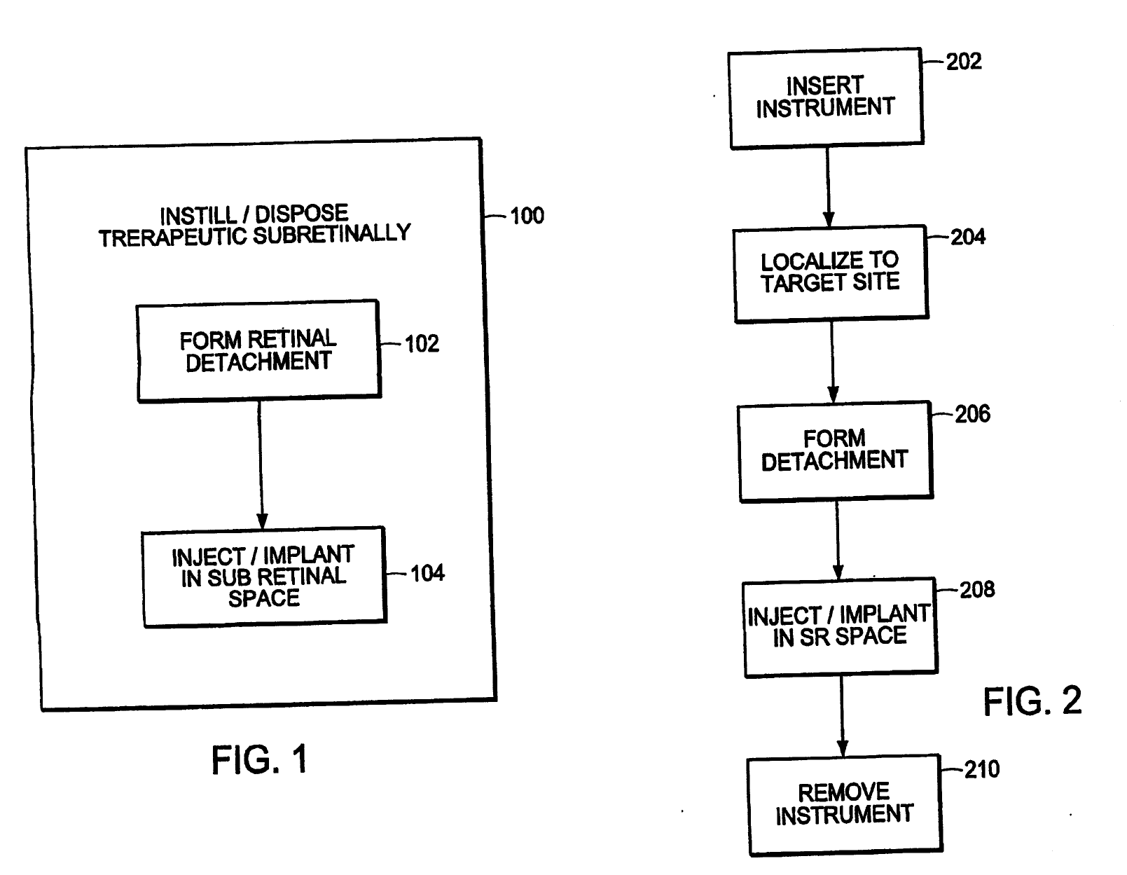

According to the present invention, and with reference with FIG. 1, such administering or delivery of the therapeutic medium includes instilling or disposing a therapeutic medium, sub-retinally or into a sub-retinal space (Step 100). In more particular embodiments, such a therapeutic medium is instilled or disposed sub-retinally or in a sub-retinal space that is proximal to a given site or locus of particular tissues of the eye that require such treatment or are an appropriate pathway for effective delivery of the therapeutic medium to tissues requiring treatment or prevention of the disease/disorder. In a more particular embodiment, such instilling or disposing is accomplished by injection and/or insertion/implantation of the therapeutic medium sub-retinally or in the sub-retinal space. In this way, the action (e.g., the pharmacodynamic action) of the therapeutic medium is localized at the choroid and the retina and also minimizes the drug action at other tissue.

Such methods according to the present invention bypass the mechanisms or barriers that limit effective delivery of such therapeutic mediums if injected directly into the vitreous, thereby permitting more sustained therapy for the target tissue(s). Moreover, locating the therapeutic medium sub-retinally (e.g., in the sub-retinal space) also reduces the side effects typically associated with the injection of drugs into the vitreous (e.g., elevated intraocular pressure). Locating the therapeutic medium sub-retinally also minimizes the loss or removal of the therapeutic medium from the eye such as expiration of the therapeutic medium via the anterior segment of the eye after being initially located or injected in the vitreous. Also, such sub-retinal locating of the therapeutic medium minimizes the need for follow up injections, as typically needed with injections into the vitreous in order to maintain an adequate therapeutic concentration within the vitreous as well as minimizing the risks attendant with such injections to the vitreous. Further, because the therapeutic medium is delivered directly to the subretinal space, it follows that higher concentrations of the medium are delivered to the choroidal vessels and retinal pigment epithelial cells as compared to intravitreal injection and intraocular implants that introduce drugs into the vitreous humor.

As used in the present invention, therapeutic medium includes any compound, agent or the like known in the art that when administered or delivered sub-retinally, is effective in obtaining a desired local or systemic physiological or pharamacological effect. More particularly, in the present invention, therapeutic medium includes, but is not limited to drugs, medicaments, antibiotics, antibacterials, antiproliferatives, neuroprotectives, anti-inflammatories (steroidal and non-sterodial), growth factors, neurotropic factors, antiangiogenics, thromobolytics or genes. Exemplary therapeutic mediums include, but are not limited to, thrombin inhibitors; antithrombogenic agents; thrombolytic agents; fibrinolytic agents; vasospasm inhibitors; calcium channel blockers; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1-3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil); immunological drugs (such as vaccines and immune stimulants); hormonal agents (such as estrogens, estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotropin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Antiproliferatives include any of a number of compounds, agents, therapeutic mediums or drugs known to those skilled in the art that inhibit the proliferation of cells. Such compounds, agents, therapeutic mediums or drugs include, but are not limited to, 5-fluorouracial, taxol, rapamycin, mitomycine C and cisplatin.

Neuroprotectives include any of a number of compounds, agents, therapeutic mediums or drugs known to those skilled in the art that guard or protect against neurotoxicity; the quality of exerting a destructive or poisonous effect upon nerve tissue. Such compounds, agents, therapeutic mediums or drugs include, but are not limited to, lubezole.

Anti-inflammatories include any of a number of compounds, agents, therapeutic mediums or drugs known to those skilled in the art, either steroidal or non-steroidal, and generally characterized has having the property of counteracting or suppressing the inflammatory process. Non-steroidal inflammatory drugs or compounds comprise a class of drugs which shares the property of being analgesic, antipyretic and anti-inflammatory by way of interfering with the synthesis of prostaglandins. Such non-steroidal anti-inflammatories include, but are not limited to, indomethacin, ibuprofen, naxopren, piroxicam and nabumetone.

Such anti-inflammatory steroids contemplated for use in the methodology of the present invention, include those described in U.S. Pat. No. 5,770,589, the teachings of which are incorporated herein by reference. In an exemplary embodiment, an anti-inflammatory steroid contemplated for use in the methodology of the present invention is triamcinolone acetonide (generic name). Corticosteroids contemplated for use in the methodology of the present invention include, for example, triamcinolone, dexamethasone, fluocinolone, cortisone, prednisolone, flumetholone, and derivatives thereof (See also U.S. Pat. No. 5,770,589).

Other anti-inflammatories or anti-inflammatory factors contemplated for use in the present invention include antiflammins (see, e.g. U.S. Pat. No. 5,266,562, incorporated herein by reference in its entirety), beta-interferon (IFN-.beta.), alpha-interferon (IFN-.alpha.), TGF-beta, interleukin-10 (IL-10), and glucocorticoids and mineralocorticoids from adrenal cortical cells. It should be noted that certain biologically active materials can have more than one activity. For example, it is believed that IFN-.alpha. and IFN-.beta. have activities as both anti-inflammatory molecules and as anti-angiogenic molecules. In exemplary embodiments, the dosage of anti-inflammatory factors being delivered to the sub-retinal space is contemplated as being in a dosage range of 50 pg to 500 ng, preferably 100 pg to 100 ng, and most preferably 1 ng to 50 ng per eye per patient per day.

As is known to those skilled in the art, growth factors is a collective term originally used to refer to substances that promote cell growth and is now loosely used to describe molecules that function as growth stimulators (mitogens) but also as growth inhibitors (sometimes referred to as negative growth factors), factors that stimulate cell migration, or as chemotactic agents or inhibit cell migration or invasion of tumor cells, factors that modulate differentiated functions of cells, factors involved in apoptosis, factors involved in angiogenesis, or factors that promote survival of cells without influencing growth and differentiation. In the present invention, such growth factors include, but are not limited to, pigment epithelium derived factor and basic fibroblast growth factor.

As is known to those skilled in the art, neurotropic factors is a general term used to describe growth factors and cytokines that can enhance neuronal survival and axonal growth and that regulate synaptic development and plasticity in the nervous system. In the present invention, such growth factors include, but are not limited to, ciliary neurotrophic factors and brain-derived neurotrophic factors.

Antiangiogenics include any of a number of compounds, agents, therapeutic mediums or drugs known to those skilled in the art that inhibit the growth and production of blood vessels, including capillaries. Such compounds, agents, therapeutic mediums or drugs include, but are not limited to, anecortave acetate and anti VEGF antibody. Other antiangiogentics or anti-angiogenic factors contemplated for use with the methodology of the present invention include vasculostatin, angiostatin, endostatin, anti-integrins, vascular endothelial growth factor inhibitors (VEGF-inhibitors), platelet factor 4, heparinase, and bFGF-binding molecules. The VEGF receptors Flt and Flk are also contemplated. When delivered in the soluble form these molecules compete with the VEGF receptors on vascular endothelial cells to inhibit endothelial cell growth. VEGF inhibitors may include VEGF-neutralizing chimeric proteins such as soluble VEGF receptors. See Aiello, PNAS, 92, 10457 (1995). In particular, they may be VEGF-receptor-IgG chimeric proteins. Another VEGF inhibitor contemplated for use in the present invention is antisense phosphorothiotac oligodeoxynucleotides (PS-ODNs). In exemplary embodiments, the dosage of anti-angiogenic factors being delivered to the sub-retinal space is contemplated as being in a dosage range of 50 pg to 500 ng, preferably 100 pg to 100 ng, and most preferably 1 ng to 50 ng per eye per patient per day.

Thrombolytics, as is known to those skilled in the art include any of a number of compounds, agents, therapeutic mediums or drugs that dissolve blot clots, or dissolve or split up a thrombous. Such thrombolytics include, but are not limited to, streptokinase, tissue plasminogen activator or TPA and urokinase.

Other factors contemplated for use in the present invention for retarding cell degeneration, promoting cell sparing, or promoting new cell growth include neurotrophin 4/5 (NT4/5), cardiotrophin-1 (CT-1), ciliary neurotrophic factor (CNTF), glial cell line derived neurotrophic factor (GDNF), nerve growth factor (NGF), insulin-like growth factor-1 (IGF-1), neurotrophin 3 (NT-3), brain-derived neurotrophic factor (BDNF), PDGF, neurturin, acidic fibroblast growth factor (aFGF), basic fibroblast growth factor (bFGF), EGF, neuregulins, heregulins, TGF-alpha, bone morphogenic proteins (BMP-1, BMP-2, BMP-7, etc.), the hedgehog family (sonic hedgehog, Indian hedgehog, and desert hedgehog, etc.), the family of transforming growth factors (including, e.g., TGF.beta.-1, TGF.beta.-2, and TGF.beta.-3), interleukin 1-B (IL1-.beta.), and such cytokines as interleukin-6 (IL-6), IL-10, CDF/LIF, and beta-interferon (IFN-.beta.). In exemplary embodiments, the dosage of such factors being delivered to the sub-retinal space is contemplated as being in a dosage range of of 50 pg to 500 ng, preferably 100 pg to 100 ng, and most preferably 1 ng to 50 ng per eye per patient per day.

Modified, truncated, and mutein forms of the above-mentioned molecules are also contemplated. Further, active fragments of these growth factors (i.e., those fragments of growth factors having biological activity sufficient to achieve a therapeutic effect) are also contemplated. Also contemplated are growth factor molecules modified by attachment of one or more polyethylene glycol (PEG) or other repeating polymeric moieties. Combinations of these proteins and polycistronic versions thereof are also contemplated.

The therapeutic medium/media being instilled or disposed sub-retinally or in the sub-retinal space is in any of a number of formulations including fluid solutions, solids and/or sustained release formulations or devices. In an even more particular embodiment, such instilling or disposing includes forming a local or limited retinal detachment (e.g., bleb detachment) using any of a number of devices and/or techniques known to those skilled in the art so as to define a sub-retinal space and injecting and/or implanting the therapeutic medium, in what ever form it may be, into the sub-retinal space defined by the local/limited retinal detachment.

The methodology of the present invention advantageously delivers the therapeutic medium to the target or disease site and thus the eye as compared to current systemic and intraocular routes of administration. More particularly, the methodology of the present invention allows the highest achievable drug concentration at the target or disease site, a low dosage requirement, and minimal aqueous and vitreous concentrations, thereby consequently reducing side effects (e.g., glaucoma, cataract, etc.) that can be exhibited when using current techniques.

In further embodiments, sustained releases devices of the present invention include, but are not limited to the following characteristics; flexible rods, thin films, foldable discs, biodegradable polymers with the therapeutic medium (e.g., drug) embedded within, drug eluting polymer coatings over a rigid scaffold; compressed drug “pellets” or a therapeutic medium encapsulated in a semi-permeable membrane. Also, some characteristic formulations for delivery of the therapeutic medium into the subretinal space include, but are not limited to, injectable hydrogels, cyclodextrin “solubilized” and micronized solutions.

A variety of biocompatible capsules are suitable for delivery of the therapeutics medium. Exemplary biocompatible polymer capsules contemplated for use with the methodology of the present invention includes (a) a core which contains the therapeutic medium, either suspended in a liquid medium or immobilized within a biocompatible matrix, and (b) a surrounding jacket comprising a membrane that is biocompatible and permits diffusion of the drugs, therapeutics, medicaments such as proteins, cells or small molecule pharmaceuticals, or the like to the tissues proximal the sub-retinal space. As indicated above, the core may comprise a biocompatible matrix of a hydrogel or other biocompatible matrix material that stabilizes the position of the therapeutic medium. The jacket for the capsule may be manufactured from various polymers and polymer blends including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers, and mixtures thereof.

In yet a more particular embodiment of the methodology of the present invention, and with reference to FIG. 1, the step of sub-retinal instilling or disposing the therapeutic medium (Step 100) includes forming a limited or localized retinal detachment (Step 102), thereby defining or forming a sub-retinal space and injecting and/or inserting/implanting of the therapeutic medium into the sub-retinal space formed by the retinal detachment (Step 104). The limited or local sub-retinal detachment is created in such a fashion that the detachment itself generally does not have an appreciable or noticeable long-term effect on the vision of the person.

It is understood that the amount of the therapeutic medium that is to be delivered to the treatment site is readily calculable by one of ordinary skill in the art without undue experimentation and will vary depending on the disease or disorder to be treated and the particular treatment circumstances. In addition, the amount also will depend upon the particular formulation of the therapeutic medium, such as for example, whether the therapeutic medium is a sustained release formulation and/or in a sustained release device. Further, the amount of the therapeutic medium to be delivered also takes into account the period of time expected for administration and/or treatment and/or the frequency or periodicity of such administration and/or treatment. The injection formulation also ordinarily takes into account pH, osmolarity and toxicity. In more particular embodiments, the therapeutic medium is in the form of one of a solid, a hydrogel, a solution, a composition or a liquid.

The therapeutic medium to be administered is preferably concentrated as feasible to minimize the volume to be administered sub-retinally or into the sub-retinal space. After the liquid and the therapeutic medium is administered or instilled sub-retinally, the surrounding tissues absorb the liquid and the therapeutic medium resides sub-retinally (e.g., as a solid) and diffuses or otherwise is absorbed by the surrounding tissues of the eye over time. In this way, the methods of the present invention provide a localized sub-retinal deposit of the therapeutic medium within the eye. In addition, the action of the deposit or depot of the therapeutic medium also is localized at the retina and the choroid.

In the case where the therapeutic medium is initially formed so as to be in the form of a solid, such solids can further be in the form of a capsule, a pellet, a rod, a sheet or film, or a hydrogel. Further such solids can be further configured and arranged so as to comprise a sustained release device for controllably releasing the therapeutic medium, and/or the active element(s) comprising the therapeutic medium to the tissues of the eye. Examples of sustained release devices are found in, for example, U.S. Pat. No. 5,378,475 and U.S. Pat. No. 5,773,019 the teaching sof which are incorporated herein by reference. See also the related discussion in U.S. Pat. No. 6,217,895, the teachings of which are incorporated by reference in their entirety.

The sustained release device for use in the present invention is one that can be administered, implanted or delivered sub-retinally and so as to release or deliver therapeutic medium, more particularly a therapeutic dosage of the therapeutic medium (e.g., corticosteroids and anti-inflammatory steroids), for a sustained period of time, that is for example for about 1 month to about 20 years, such as from about 6 months to about 5 years and more specifically from about 3 months to a year. In an exemplary embodiment, the sustained release device is prepared, configured and/or arranged so as to release the therapeutic medium by pseudo zero order release kinetics.

The capsule or other structure forming the solid or the sustained release device can be any suitable configuration, including cylindrical, rectangular, disk-shaped, patch-shaped, ovoid, stellate, or spherical. It is desirable, however, to use a configuration that does not tend to lead to migration of a capsule(s) or other structure from the sub-retinal space, such as spherical shapes, so as to minimize the potential for migration of the instilled therapeutic medium from the targeted tissue site.

The therapeutic medium also can include a pharmaceutically acceptable carrier or excipient and/or one or more accessory molecules which may be suitable for diagnostic or therapeutic use in vitro or in vivo. The term “pharmaceutically acceptable carrier” as used herein encompasses any of the standard pharmaceutical carriers, such as a phosphate buffered saline solution, water, and emulsions, such as an oil/water or water/oil emulsion, and various types of wetting agents. The therapeutic medium also can include stabilizers and preservatives. For examples of carriers, stabilizers and adjuvants, see Martin Remington's Pharm. Sci., 15th Ed. (Mack Publ. Co., Easton (1975)).

It also should be recognized, that the methodologies of the present invention are contemplated as being practiced alone, or in combination with other therapies or treatments. For example, where laser treatment of an eye is indicated, the therapeutic medium can be administered (e.g., instilled or disposed) sub-retinally before and/or after the laser treatment. In addition, it is contemplated that the therapeutic medium can comprise a mixture of active agents or therapeutic agents such as for example antibiotics, medicaments, or agents, e.g., thalidomide, being administered along with a steroid.

Most, if not all, ophthalmic diseases and disorders are associated with one or more of three types of indications: (1) angiogenesis, (2) inflammation, and (3) degeneration. Based on the indications of a particular disorder, one of ordinary skill in the art can administer any suitable therapeutic medium molecule from the three groups at a therapeutic dosage. The following describes some ophthalmic diseases and disorders and a form of treatment therefore. It should be recognized however, that the following is by way of illustration and is not intended to limit the methodologies of the present invention to a particular technique or therapeutic medium for treatment of an eye disease or disorder.

Diabetic retinopathy, for example, is characterized by angiogenesis. This invention contemplates treating diabetic retinopathy by delivering one or more anti-angiogenic factors into the sub-retinal space. It also is desirable to co-deliver one or more neurotrophic factors also to the sub-retinal space.

Uveitis involves inflammation. The present invention contemplates treating uveitis by instilling or disposing one or more anti-inflammatory factors in the sub-retinal space.

Retinitis pigmentosa, by comparison, is characterized by retinal degeneration. The present invention contemplates treating retinitis pigmentosa by instilling or disposing one or more neurotrophic factors in the sub-retinal space.

Age-related macular degeneration involves both angiogenesis and retinal degeneration and includes, but is not limited to, dry age-related macular degeneration, exudative age-related macular degeneration, and myopic degeneration. The present invention contemplates treating this disorder by instilling or disposing in the sub-retinal space one or more neurotrophic factors and/or one or more anti-angiogenic. More particularly, the methodology contemplates instilling or disposing a corticosteriod in the sub-retinal space.

Glaucoma is characterized by increased ocular pressure and loss of retinal ganglion cells. Treatments for glaucoma contemplated in the present invention include delivery of one or more neuroprotective agents that protect cells from excitotoxic damage. Such agents include N-methyl-D-aspartate (NMDA) antagonists, cytokines, and neurotrophic factors.

As noted above, administration of the therapeutic medium is not limited to those uses involving the diagnosed existence of a disorder or disease. The methodology of the present invention also contemplates prophylactic administration of a therapeutic medium. For example, in more than 50% of cases where AMD occurs in one eye, it will subsequently occur in the unaffected eye within a year. In such cases, prophylactic administration of a therapeutic medium such as a steroid into the unaffected eye may prove to be useful in minimizing the risk of, or preventing, AMD in the unaffected eye.

As indicated herein, in a more particular aspect of the present invention, steroids, including anti-inflammatory steroids and corticosteroids, are disposed or instilled in the sub-retinal space. Such steroids shall be in any of a number of forms known to those skilled in the art appropriate for the distribution of the drug to the tissues of the eye that are proximal to the targeted sub-retinal site or the sub-retinal space. In more particular embodiments, the steroids are in the form of one of a solid, a hydrogel, a solution, composition or a liquid.

For example, anti-inflammatory steroids are typically crystalline and are administered in a liquid such as distilled water or a balanced salt solution with a minimum of carriers or adjuvants. A depot pharmaceutical composition, however, that includes an effective or therapeutic amount of an anti-inflammatory steroid together with a pharmaceutically and ophthalmologically acceptable carrier, diluent and/or excipient is contemplated for use in the present invention. When triamcinolone acetonide is to be the anti-inflammatory steroid, such a preparation can be made up by using Kenacort-A40 (registered trade mark) (Squibb) as the anti-inflammatory steroid. Further, suitable pharmaceutically acceptable salts of this compound can be used, for example, the acetate of triamcinolone acetonide.

The anti-inflammatory steroid to be administered is preferably concentrated as feasible to minimize the volume to be administered sub-retinally or into the sub-retinal space. In an exemplary embodiment, the dosage of the steroid is between about 10 μg and about 500 μg. This dosage range is applicable to each of the three following stages of macular degeneration, namely: early onset macular degeneration, atrophic macular degeneration (AMD) and neovascular macular degeneration (NMD).

After the liquid and anti-inflammatory steroid is administered or instilled sub-retinally, the surrounding tissues absorb the liquid and the steroid resides sub-retinally as a solid and diffuses or otherwise is absorbed by the surrounding tissues of the eye over time. In this way, the methods of the present invention provide a localized sub-retinal deposit of steroids within the eye. In addition, the action of the deposit or depot of steroids is also localized at the retina and the choroid. It should be recognized that while the foregoing is described in connection with anti-inflammatory steroids, the foregoing is illustrative and shall not be construed as limiting or restricting the described techniques to anti-inflammatories as other steroids are contemplated for use with the above-described technique.

In the case where the steroids are initially formed so as to be in the form of a solid, such solids can further be in the form of a capsule, a pellet, a rod, a sheet or film, or a a hydrogel. Further such solids can be further configured and arranged so as to comprise a sustained release device for controllably releasing the steroid, and/or the active element(s) comprising the steroids to the tissues of the eye as herein described above.

The sustained release device for use in the present invention is one that can be administered, implanted or delivered sub-retinally and so as to release or deliver steroids more particularly a therapeutic dosage of steriods, such as corticosteroids and anti-inflammatory steroids, for a sustained period of time, that is for example for about 1 month to about 20 years, such as from about 6 months to about 5 years and more specifically from about 3 months to a year. In an exemplary embodiment, the sustained release device is prepare, configured or arranged so as to release the steroids by pseudo zero kinetics.

The capsule or other structure forming the solid or the sustained release device can be any suitable configuration, including cylindrical, rectangular, disk-shaped, patch-shaped, ovoid, stellate, or spherical. It is desirable, however, to use a configuration that does not tend to lead to migration of a capsule(s) or other structure from the sub-retinal space, such as spherical shapes, so as to minimize the potential for migration of the instilled steroids from the targeted tissue site.

As indicated herein, the methodologies of the present invention are contemplated as being practiced alone, or in combination with other therapies or treatments. Thus, for example, where laser treatment of an eye is indicated, the steroid can be administered (e.g., instilled or disposed) sub-retinally before and/or after the laser treatment. In addition, it is contemplated that antibiotics, other therapeutics, medicaments, or agents, e.g., thalidomide, can be administered along with the steroid(s). As noted above, administration of a therapeutic medium is not limited to those uses involving the diagnosed existence of a disorder or disease; as such the methodology of the present invention according to this aspect of the present invention also contemplates prophylactic administration of the steroid(s).

Now referring to FIG. 2, there is shown a flow diagram of an eye treatment methodology according to another embodiment of the present invention, which methodology includes inserting a delivery device or delivery instrument into the eye to be treated (Step 202). The instrument being inserted can be any of a number of instruments known to those skilled in the art that can be used to form a retinal detachment. More particularly, the instrument is configured and arranged so as to be capable of forming a limited or localized retinal detachment and to minimize the area of the retinal detachment such that there is no long-term apparent loss in visual acuity.

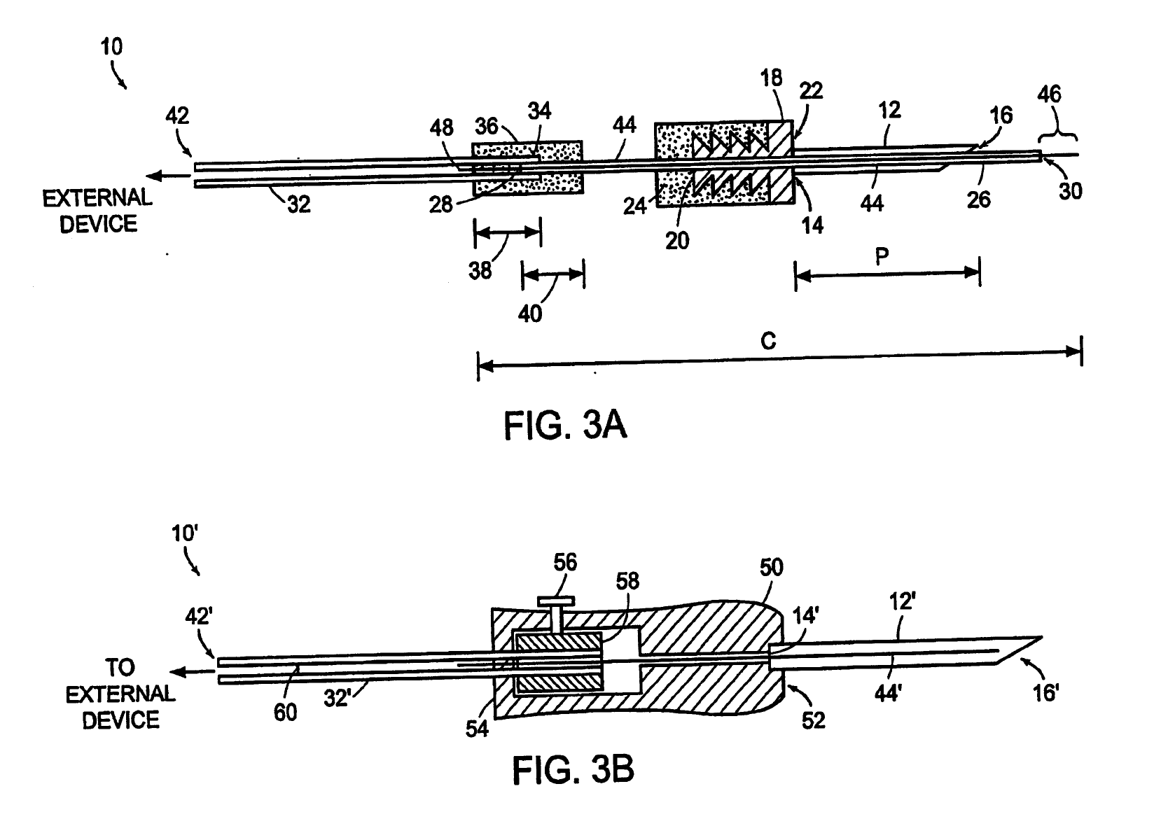

In illustrative, exemplary embodiments, the instrument being inserted to deliver the therapeutic medium sub-retinally and/or to create a localized or limited retinal detachment includes the delivery instruments or devices 10, 10′ as shown in FIGS. 3A,B. Reference also should be made to U.S. Ser. No. 09/888,079 (now U.S. Patent Application Publication U.S. 2002/0198511A1), the teachings of which are incorporated herein in their entirety for further details of the delivery devices 10, 10′ illustrated in FIGS. 3A,B and not described herein.

Referring now to FIG. 3A, there is shown an illustrative delivery device 10 that includes a piercing member 12, which has a proximal end 14 and a distal end 16, with a lumen defined there between. The distal end 16 of the piercing member 12 is pointed (e.g., beveled) to allow for the piercing member to pierce and penetrate a target/treatment site as will be described below. In exemplary embodiments, the piercing member 12 is configured such that the outer diameter is about 25 gauge (0.5 millimeter) or less.

The proximal end 14 of the piercing member 12 is connected to a first connection element 18 having a proximal end 20 and a distal end 22 and a lumen defined therebetween. The diameter of the first connection element lumen should be substantially identical to that of the piercing member lumen such that these lumens are substantially longitudinally aligned to create a fluid tight passageway when connected. Optionally, but preferably, a seal 24 is connected to the first connection element 18, and substantially surrounds at least a portion of the first connection member lumen in order to further enhance the integrity of the fluid tight passageway.

The fluid tight passageway is sized to accommodate a rigid member 26, which adds physical stability to the device 10. The rigid member 26 has a proximal end 28 and a distal end 30, and a lumen defined therebetween. The distal end 30 of the rigid member 26 extends distal to the distal end 16 of the piercing member, while the proximal end 28 of the rigid member extends proximate to the seal 24. A cannula 44 is disposed within the rigid member 26, and preferably is physically connected to the rigid member 26 such that any distal-to-proximal or proximal-to-distal movement of the rigid member effects corresponding movement of the cannula, and such that any distal-to-proximal movement of the cannula effects corresponding movement of the rigid member.

As shown in FIG. 3A, the rigid member 26 extends into a quantity of tubing 32 such that the proximal end 28 of the rigid member is proximal to the distal end 34 of the tubing. A second connection element 36 preferably surrounds a distal portion 38 of the tubing 32 and a proximal portion 40 of the rigid member 26 so as to maintain the connection between the tubing and rigid member. The tubing 32 includes a proximal end 42, which is in communication with an external supply or withdrawal device (not shown) either directly or via a connection element. In this way, material (e.g., fluid, air, etc.) can be supplied into, or withdrawn from the tubing.

Referring now to FIG. 3B, there is shown an alternative delivery device 10′ that is similar in structure and operation to the delivery device 10 of FIG. 3A, but includes a handle 50 and does not utilize a rigid member 26. The alternative delivery device 10′ includes a piercing member 12′ substantially as described above. The proximal end 14′ of the piercing member 12′ is connected to the distal end 52 of a handle 50, which has a proximal end 54 that is connected to a quantity of tubing 32′. By virtue of its connection to both the piercing member 12′ and the tubing 32′, the handle 50 is not only effective to facilitate initial and continued grasping of the device 10′, but also to stabilize and provide support to the device 10′. Each of the handle 50, the piercing member 12′ and the tubing 32′ has a lumen defined therebetween, thus defining a pathway between the distal end 16′ of the piercing member and the proximal end 42′ of the tubing. A cannula is disposed within, and, preferably, connected to the lumen 60 defined within the tubing 32′. By virtue of this connection, distal-to-proximal and proximal-to-distal movement of the tubing 32′ will result in corresponding movement of the cannula 44′, and vice versa.

The handle 50 also includes an actuating element 56 that sits within a slot (not shown) or other opening. The actuating element 56 is in communication with a housing 58, which is in communication with the distal end 34′ of the tubing 32′ as shown in FIG. 3B. By virtue of this arrangement, distal-to-proximal or proximal-to-distal movement of the actuating element 56 within the slot causes substantially corresponding movement of the housing, which, in turn, causes substantially corresponding movement of the tubing and, therefore, of the cannula 44′ as well.

Referring back to only FIG. 2, in further embodiments, the step of inserting (Step 202) further includes inserting a portion of the delivery instrument or device, including but not limited to the delivery devices 10,10′ illustrated in FIGS. 3A,B, into the eye in a minimally invasive manner. This methodology also yields a technique that can be implemented in an outpatient clinic setting. According to this further embodiment, a delivery instrument or device is provided, a portion of which is configured and arranged such that when the instrument is inserted into the eye, the opening formed in the sclera to receive the instrument is small enough so as to not require sutures to seal or close the opening in the sclera. In other words, the opening is small enough that the wound or opening is self-sealing, thereby preventing the aqueous humor from leaking out of the eye.

In addition, the step of inserting further includes inserting the insertable portion of the delivery instrument or device transconjunctivally so the operable end thereof is within the vitreous. In this regard, transconjunctival shall be understood to mean that the instrument's operable end is inserted through both the conjunctiva and through the sclera into the vitreous. More particularly, inserting the insertable portion that forms an opening in the sclera and the conjunctiva that is small enough so as to not require sutures or the like to seal or close the opening in the sclera. In conventional surgical techniques for the posterior segment of the eye, the conjunctiva is routinely dissected to expose the sclera, whereas according to the methodology of this embodiment, the conjunctiva need not be dissected.

Consequently, when the instrument is removed from the eye (step 210), the surgeon does not have to seal or close the opening in the sclera with sutures to prevent leaking of the aqueous humor because as indicated above such an opening or wound in the sclera is self-sealing. In addition, with the transconjunctical approach, the surgeon does not have to deal with reattaching the dissected conjunctiva. Thus, further simplifying the surgical procedure as well as reducing if not eliminating the suturing required under the surgical procedure.

After the insertable portion of the instrument is inserted into the eye, the operable end thereof is localized to the targeted site (Step 204) including the tissues that are being targeted for treatment. As is known to those skilled in the art, surgical personnel typically mount a lens assembly (not shown) onto the cornea of the eye in accordance with known and accepted practices and techniques. This lens assembly is provided so that the surgeon can view the interior of the eye as well as any instruments inserted therein. In addition, a light-transmitting apparatus as is known in the art also is inserted into the vitreous so as to be capable of providing a source of light therein for the surgeon. Accordingly, the surgeon would determine the positioning of the operable end of the instrument by viewing the interior of the eye using the lens assembly and being illuminated by the light transmitting apparatus.

After localizing the operable end of the instrument to the target site, for example the surface of the retina proximal the target site, the surgeon or medical personnel forms the limited retinal detachment (Step 206). In an illustrative exemplary embodiment, the surgeon forms the limited retinal detachment by injecting a fluid, such as liquid or gas, from the instrument's operable end. More specifically, the fluid is injected from the instrument's operable end in such a manner that the injected fluid is disposed between the retina and the choriod thereby causing the retina to detach therefrom. In more specific embodiments, the instrument's operable end is positioned such that the stream of fluid flowing from the operable end of the instrument is directed towards the targeted site of the retina and the stream of fluid pierces the retina and flows beneath the retina.

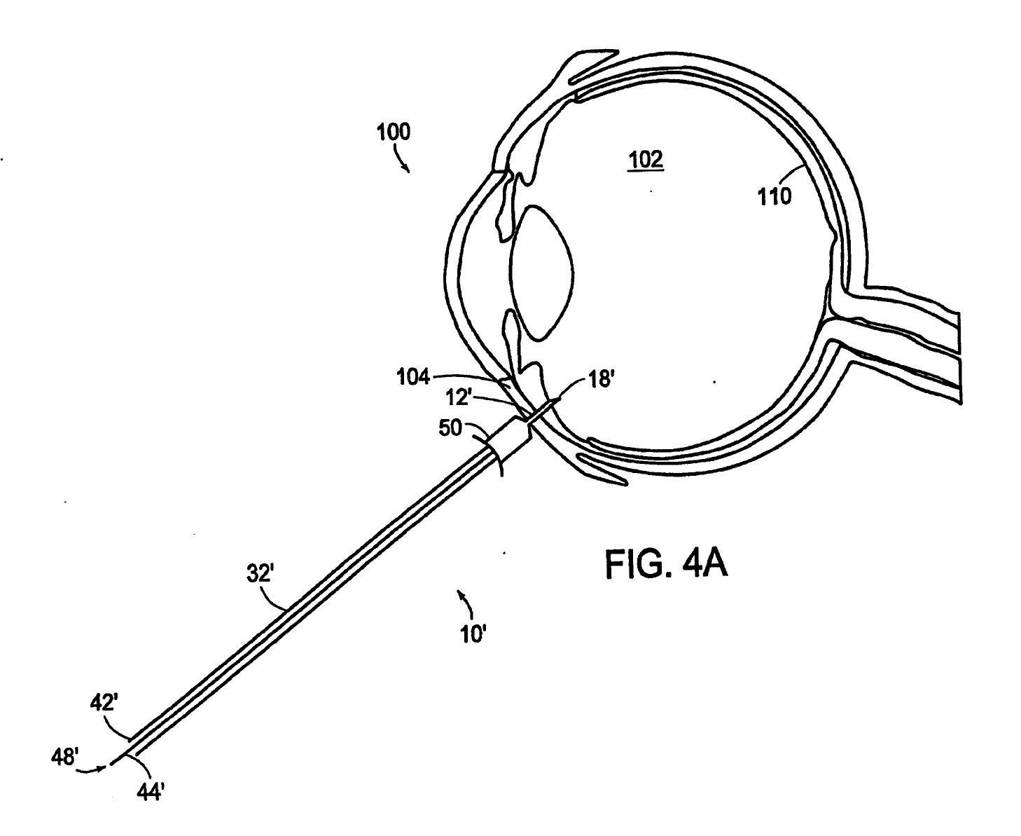

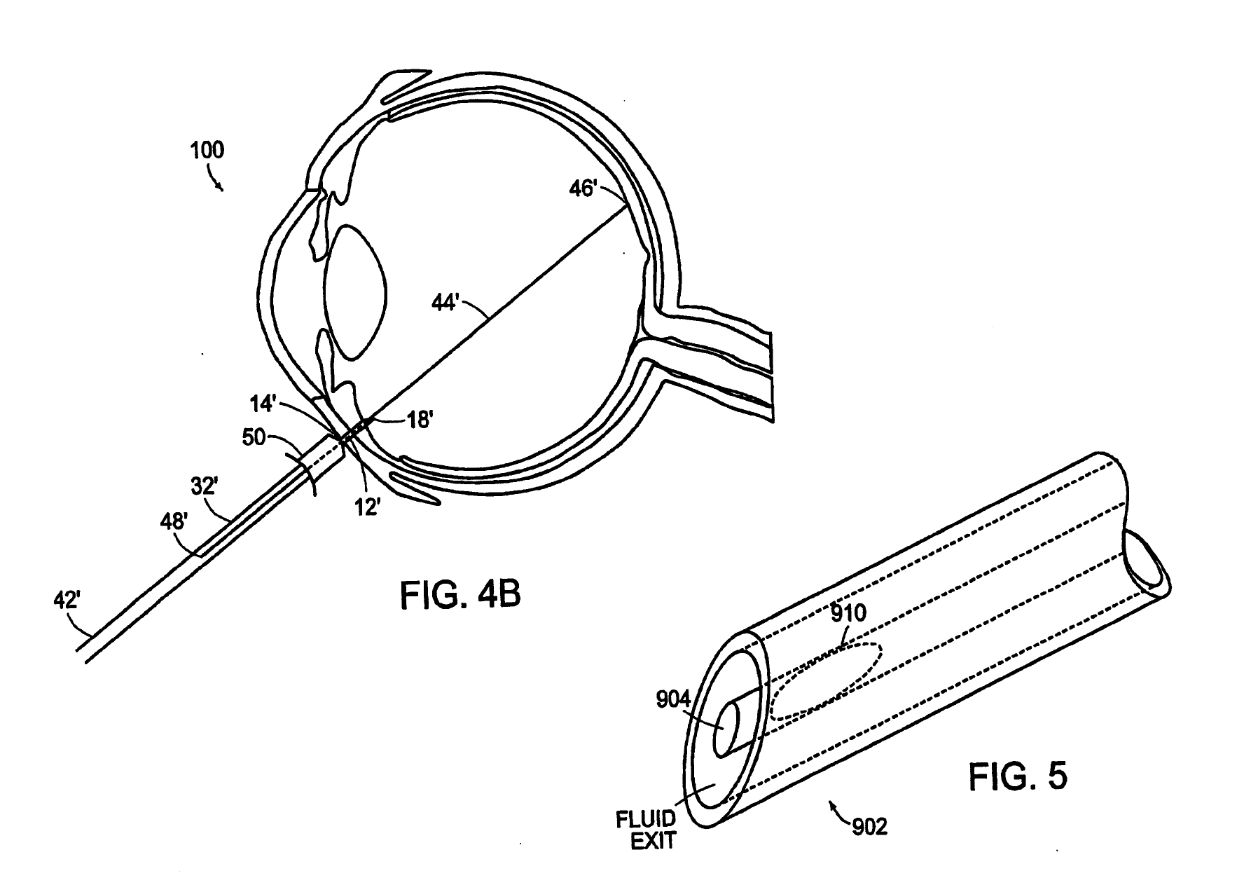

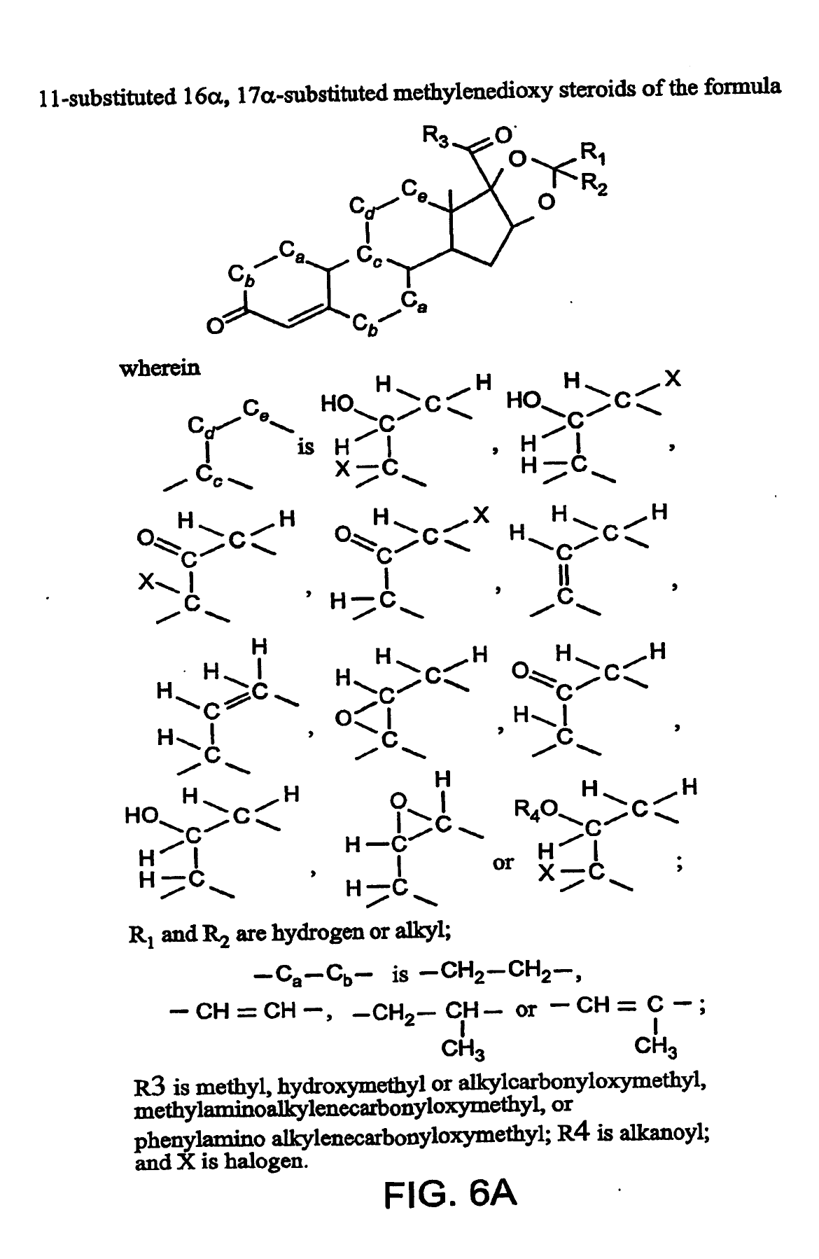

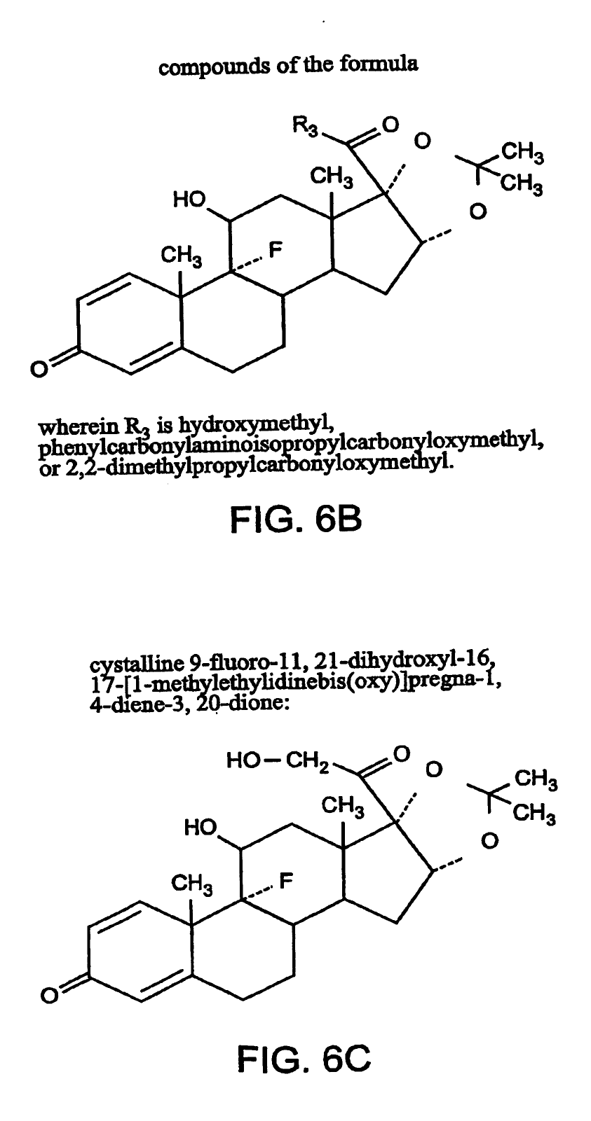

In the case of the delivery devices 10, 10′ illustrated in FIGS. 3A,B, there is illustrated in FIGS. 4A,B inserting the instrument/device so a portion thereof including the operable end is disposed in the eye and localizing the operable end of the device to the target site. Reference also should be made to U.S. Ser. No. 09/888,079 (now U.S. Patent Application Publication U.S. 2002/0198511A1), the teachings of which are incorporated herein in their entirety for further details of the inserting and localizing not illustrated in FIGS. 4A,B and not described herein.

The sharp distal end 18′ of the piercing member 12′ is localized to a desired location on the surface of the conjunctiva or the sclera 104 of the eye 100. A pressure or force is applied to the device 10′ such that the sharp distal end 18′ of the piercing member 12′ penetrates the sclera 104 of the eye 100 or both the conjunctiva and sclera of the eye and the distal end is within the vitreous humor 102 of the eye 100. This also thus creates a continuous passageway (not shown) between the device 10′ and the vitreous humor 102 of the eye 100 providing a pathway for the surgeon to gains access to the vitreous humor.

The piercing member 12′ also has a length such that once its proximal end 16′ is in contact with a portion of the outer periphery of the sclera or the conjunctiva of the eye, the distal end 18′ of the piercing member is within the vitreous humor 102 of the eye 100. Once inserted the piercing member 12′ can be angled by gently tilting or manipulating any portion of the device that lies outside of the eye 100. In this way, the device 10′ can be localized to multiple target sites within the eye without necessitating multiple, separate insertions of the device into the eye.

Once a passageway into the eye 100 is thus created, the cannula 44′ and attached tubing 32′ (or, in the case of the device 10 of FIG. 3A, the rigid member 26 with attached cannula 44 positioned therewithin) is advanced into and through the device 10′ and localized to a treatment/target site. As illustrated in FIG. 4B, the target site is the retina 110 of the eye 100. The cannula 44′ is guided through the device 10′ until a distal portion 46′ of the cannula emerges from the guiding member 12′, and into the vitreous humor 102 and the cannula is further advanced within the eye 100 until the distal portion 46′ of the cannula enters the retina 110.

An operator (e.g., surgeon) of the device 10′ is able to determine that the distal portion 46′ of the cannula 44′ has entered, but not traveled completely through, the retina 48 by virtue of techniques generally known in the art. For example, once an operator estimates that the distal portion 46′ of the cannula is approaching the retina, s/he can inject an agent through the cannula 44′. In order to simplify this estimation, the cannula 44′ can include one or more markings that serve as visual and/or tactile indicators of the relative position of the cannula with respect to the retina. If, following this injection, the formation of a retinal detachment is observed, the operator can safely deduce that the distal portion 46′ of the cannula 44′ has entered, and still remains within, the retina 110 and can halt the distal advancement of the cannula.

Now referring back to only FIG. 2, after forming the localized or limited retinal detachment (e.g., a bleb detachment), the therapeutic medium is injected or implanted in the sub-retinal spaced defined by the limited retinal detachment (Step 208). In the case, where the therapeutic medium is in a liquid form or formulation, the instrument forming the retinal detachment can be used to inject the therapeutic medium into the retinal detachment. Alternatively, a fluid including the therapeutic medium can be used to form the retinal detachment and thereby simultaneously form the detachment and inject the therapeutic medium. Thus, the forming of the detachment (step 208) and the injection of the therapeutic medium (step 210) are performed essentially simultaneously, thereby further simplifying the procedure or process.

In the case where the therapeutic medium is in a solid or implantable form or formulation and the operable end 902 of the instrument is further configured and arranged so to include a cannula 904 or lumen, the therapeutic medium in its implantable form 910 such as a capsule, rod or sheet is disposed the cannula or lumen prior to it being deployed there from sub-retinally. An exemplary arrangement for the operable end 902 is shown illustratively in FIG. 5. Thus, after forming the limited retinal detachment, the surgeon or medical personnel manipulates the instrument so that the therapeutic medium in its implanted form 910 is dispensed from the end of the cannula 904 in the instrument's operable end 902 into the sub-retinal space formed by the limited retinal detachment. Alternatively, the surgeon or medical personnel can manipulate the implantable form of the therapeutic medium so as to insert the therapeutic medium at the same time as forming the retinal detachment. Such dispensing can be accomplished by mechanical action on the implantable form of the drug (e.g., a rod acting on the capsule form of the drug) or by fluid or hydraulic action on the implantable form.

After completing such injection or implanting, the instrument is removed from the eye (Step 210) and any further actions are performed that may be required to seal or close the opening formed in the eye to insert the instrument. For example, in the case where an incision was made in the sclera to insert the instrument, sutures would be used to close the incision. In addition, if the particular technique also involved dissection of the conjunctiva, the conjunctiva would be re-attached to the eye. As indicated herein, if the technique used to form the opening yields an opening in the sclera small enough so as to be self sealing, suturing may not be required and for the transconjunctival technique, re-attachment of the conjunctiva should not be required.

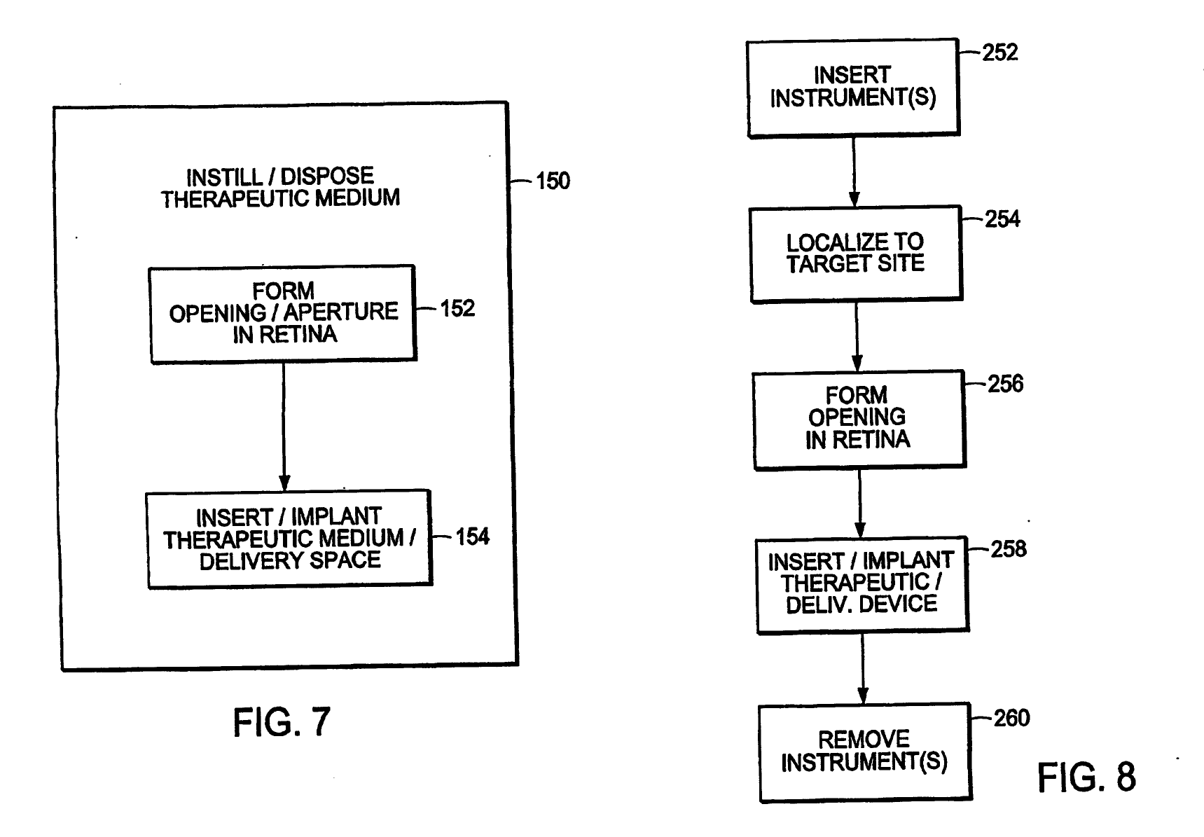

There is shown in FIG. 7 yet another embodiment of the methodology of the present invention, where the step of sub-retinal instilling or disposing the therapeutic medium (Step 150) includes accessing the area of region between the retina and the choroids, hereinafter subretinal region, (step 152) and injecting and/or inserting/implanting the therapeutic medium into accessed area or region the sub-retinal space formed by the retinal detachment (Step 154). Reference shall be made to the foregoing discussion regarding FIGS. 1-2 for further details of the therapeutics, the delivery devices, treatment methods and types of diseases and/or disabilities treatable using the methodologies of the present invention. Such accessing of the sub-retinal region is generally accomplished with the formation of limited or local sub-retinal detachment. In more particular embodiments, the retina is pierced or penetrated by a piercing device (e.g., a small gauge needle) thereby providing access to the sub-retinal region and thereafter the therapeutic medium is inserted, injected or implanted in the sub-retinal region.

In more particular embodiments, the therapeutic medium is initially formed so as to be in the form of a solid, such solids can further be in the form of a capsule, a pellet, a rod, a sheet or film, or a hydrogel. Further such solids can be further configured and arranged so as to comprise a sustained release device or delivery device for controllably releasing the therapeutic medium, and/or the active element(s) comprising the therapeutic medium to the tissues of the eye. After the therapeutic medium is administered or instilled sub-retinally, the surrounding tissues absorb any liquid that may heave been used in connection with the insertion/implantation such that the therapeutic medium resides sub-retinally (e.g., as a solid) and diffuses or otherwise is absorbed by the surrounding tissues of the eye over time. In this way, the methods of the present invention provide a localized sub-retinal deposit of the therapeutic medium within the eye. In addition, the action of the deposit or depot of the therapeutic medium also is localized at the retina and the choroid. As indicated herein, such a sustained release device or delivery device of the present invention include, but are not limited to the following characteristics; flexible rods, thin films, foldable discs, biodegradable polymers with the therapeutic medium (e.g., drug) embedded within, drug eluting polymer coatings over a rigid scaffold; compressed drug “pellets” or a therapeutic medium encapsulated in a semi-permeable membrane.

In more particular embodiments, the configuration, arrangement or shape of the therapeutic medium and/or the device for delivering the therapeutic medium is set so to be capable of being passing through the opening or through aperture formed in the retina and being inserted or implanted in the subretinal region without the need to first form a localized retinal detachment. The therapeutic medium to be administered is preferably concentrated as feasible to minimize the volume to be administered sub-retinally. In addition, the configuration, arrangement or shape of the therapeutic medium and/or the device delivering the therapeutic medium is established such that the retinal detachment resulting from the insertion or implantation of the sustained release device or delivery device subretinally does not have an appreciable or noticeable long-term effect on the vision of the person.

Now referring to FIG. 8, there is shown a flow diagram of an eye treatment methodology according to yet another embodiment of the present invention, which methodology includes inserting a device or instrument into the eye to be treated (Step 252). The instrument being inserted can be any of a number of instruments known to those skilled in the art that can be used to pierce the tissues of the retina and forming an opening or through aperture therein so as to provide access to the area or region between the retina and choroids. In a particular illustrative embodiment of the present invention, the opening or through aperture is formed by a small gauge needle that is disposed within the vitreous and manipulated by the surgeon so as to pierce the tissues of the retina. For example, a surgeon can use micro-forceps as is known to those skilled in the art that the surgeon would use to grip and manipulate the needle.

In another illustrative embodiment a sustained release device or delivery device is in the formed so that it presents in cross-section a similar sized cross section as a small gauge needle, more particular a filament having such a cross-section. In more particular embodiments, one end of the device also is configured so as to allow that end to easily penetrate the tissue of the retina. In this illustrative embodiment, the surgeon would grasp and manipulate the device using the micro-forceps so the one end is aimed towards the retina.

It should be recognized that the foregoing reflects a few illustrative embodiments, however, it is with the scope of the present invention for the insertion of the device through the retina to utilize a surgical tool that is configured and arranged so as to hold the delivery device, to form the opening or through aperture in the retina and that drives, inserts or implants the delivery device into the targeted sub-retinal region.

As with the other described embodiment of the methodology of FIG. 2, the inserted instrument in what ever form is localized to the targeted site (Step 254) that includes the tissues that are being targeted for treatment. As is known to those skilled in the art, surgical personnel typically mount a lens assembly (not shown) onto the cornea of the eye in accordance with known and accepted practices and techniques. This lens assembly is provided so that the surgeon can view the interior of the eye as well as any instruments inserted therein. In addition, a light-transmitting apparatus as is known in the art also is inserted into the vitreous so as to be capable of providing a source of light therein for the surgeon. Accordingly, the surgeon would determine the positioning of the operable end of the instrument by viewing the interior of the eye using the lens assembly and being illuminated by the light transmitting apparatus.

After localizing the operable end of the instrument to the tissues of the retina proximal the target site, the surgeon manipulates the instrument to penetrate or pierce the tissues of the retina as herein described (Step 254). As indicated hereinabove, this action preferably creates or forms an opening or through aperture in the retina of small diameter that provides access the area or region between the retina and the choroids. Preferably the opening or through aperture created or formed by such action generally does not have an appreciable or noticeable long-term effect on the vision of the person.

After forming the opening or aperture (Step 254), the surgeon then manipulates the form the therapeutic medium is in so that the form of the therapeutic medium is passed through the opening in the tissues of the retina and slide between the tissues of the choroid and the retina. In more particular embodiments, the therapeutic medium is provided in the form of a sustained release device or other delivery device and the sustained release device or delivery device is manipulated by the surgeon so as it passes through the opening or aperture in the tissues of the retina and so it is slide subretinally between the tissues of the retina and the choroids. After completion of the insertion/implantation of the therapeutic medium, the surgeon removes the surgical instruments from the vitreous (Step 260). As indicated herein, the process of inserting the instruments into the vitreous and removal preferably are accomplished using techniques whereby an opening(s) formed in the sclera for admission of the instruments into the vitreous is self-sealing. In addition, the technique used for inserting the instruments into the vitreous also is more particularly a transconjunctival technique whereby the instruments are inserted through both of the conjunctiva and the sclera.

In further embodiments, the therapeutic medium is inserted or implanted through the retinal tissues semi-permanently or temporarily. Thus, in such further embodiments the methodology further includes inserting a withdrawal instrument (e.g., micro-forceps) into the vitreous following completion of the treatment phase and localizing the operable end of the withdrawal instrument proximal the target site, more particularly proximal the tissues containing the device. Thereafter, the surgeon manipulates the withdrawal instrument so as to withdraw the therapeutic medium, for example, withdrawing the therapeutic medium delivery device from the sub-retinal region. The therapeutic medium is withdrawn from the vitreous along with any instruments. In yet further particular embodiments, the methodology of the present invention contemplates insertion of another depot of therapeutic medium, for example insertion of another delivery device with a fresh charge of therapeutic medium, into the subretinal region following such withdrawal of the used device or therapeutic medium.

Although a preferred embodiment of the invention has been described using specific terms, such description is for illustrative purposes only, and it is to be understood that changes and variations may be made without departing from the spirit or scope of the following claims.

1. A method for administering a therapeutic medium to a posterior segment of an eye, the method comprising the step of:
instilling the therapeutic medium sub-retinally.

2. The administering method of claim 1, wherein said step of instilling includes localizing the action of the therapeutic medium at the choroid and the retina and minimizing action at other tissues of the eye.

3. The administering method of claim 1, wherein the therapeutic medium being administered is one of a drug, medicament, antiproliferative, neuroprotective, steroidal anti-inflammatory, non-sterodial anti-inflammatory, growth factor, neurotropic factor, antiangiogenic, thromobolytic or a gene.

4. The administering method of claim 3, wherein the steroidal anti-inflammatrory is selected from the group consisting of triamcinolone, dexamethasone, fluocinolone, cortisone, prednisolone, flumetholone, and derivatives thereof.

5. The administering method of claim 1, wherein a disease state to be treated is selected from the group consisting of ocular neovascularization, ocular inflammation and retinal degeneration.

6. The administering method of claim 1, wherein said step of instilling includes forming a depot of a drug between the choroid and the retina.

7. The administering method of claim 1, wherein said step of instilling includes one of injecting or implanting the therapeutic medium sub-retinally.

8. The administering method of claim 7, wherein said step of instilling includes injecting a solution including the therapeutic medium between the choroid and the retina.

9. The administering method of claim 1, wherein said step of instilling includes instilling the therapeutic medium in a sub-retinal space.

10. The administering method of claim 1, wherein the therapeutic medium comprises a sustained release device and wherein said step of instilling includes implanting the sustained release device in a sub-retinal space.

11. The administering method of claim 1, wherein said step of instilling includes:
creating a localized retinal detachment to define a sub-retinal space; and
disposing the therapeutic medium in the sub-retinal space formed by the localized retinal detachment.

12. The administering method of claim 11, wherein said disposing includes one of injecting or implanting the therapeutic medium in the sub-retinal space formed by the retinal detachment.

13. The administering method of claim 12, wherein said injecting includes injecting a solution including the therapeutic medium in the sub-retinal space.

14. The administering method of claim 12, wherein the therapeutic medium comprises a sustained release device and wherein said step of implanting includes implanting the sustained release device in the sub-retinal space.

15. The administering method of claim 14, wherein the sustained release device is configured to provide a sustained sub-retinal release of the therapeutic medium.

16. The administering method of claim 10, wherein the sustained release device is configured to provide a sustained sub-retinal release of the therapeutic medium.

17. The administering method of claim 15, wherein the therapeutic medium is one of a drug, medicament, antiproliferative, neuroprotective, steroidal anti-inflammatory, non-sterodial anti-inflammatory, growth factor, neurotropic factor, antiangiogenic, thromobolytic or a gene.

18. The administering method of claim 17, wherein the steroidal anti-inflammatory is selected from the group consisting of triamcinolone, dexamethasone, fluocinolone, cortisone, prednisolone, flumetholone, and derivatives thereof.

19. The administering method of claim 11, wherein a disease state to be treated is selected from the group consisting of ocular neovascularization, ocular inflammation and retinal degeneration.

20. A method for treating an eye, comprising the step of:
administering a therapeutic medium to a posterior segment of an eye sub-retinally.

21-24. (canceled)

25. The eye treatment method of claim 20, wherein said step of administering includes:
forming a sub-retinal space; and
disposing the therapeutic medium in the sub-retinal space.

26. The eye treatment method of claim 24, wherein said step of disposing includes:
injecting a solution including the therapeutic medium into the sub-retinal space.

27. The eye treatment method of claim 20, further comprising the step of controllably releasing the over time the sub-retinally administered therapeutic medium.

28. The administering method of claim 1, further comprising the step of controllably releasing over time the sub-retinally instilled therapeutic medium.

28A. (canceled)

29. A method for administering a steroid to a posterior segment of an eye, the method comprising the step of:
instilling the steroid sub-retinally.

30. The administering method of claim 29, wherein said step of instilling includes localizing the action of the steroid at the choroid and the retina and minimizing action at other tissues of the eye.

31-44. (canceled)

45. A method for treating an eye, comprising the step of:
administering a steroid to a posterior segment of an eye sub-retinally.

46. The eye treatment method of claim 45, wherein said step of administering sub-retinally includes forming a depot of a drug between the choroid and the retina.

47-57. (canceled)
